(19) United States
(12) Patent Application Publication
(10) Pub. No.: US 2019/0365948 A1
(43) Pub. Date: Dec. 5, 2019

(54) LYOPHILIZED PLACENTAL COMPOSITE SHEET AND USES THEREOF

(71) Applicant: AZIYO BIOLOGICS, INC., Silver Spring, MD (US)

(72) Inventors: Daniel DEEGAN, Silver Spring, MD (US); Frank FAN, Richmond, CA (US); Dana Sue YOO, Falls Church, VA (US)

(21) Appl. No.: 16/479,586

(22) PCT Filed: Jan. 22, 2018
(86) PCT No.: PCT/US2018/014742

§ 371 Date: Jul. 19, 2019

Related U.S. Application Data
(60) Provisional application No. 62/451,361, filed on Jan. 27, 2017.

(51) Int. Cl. A61L 27/36; A61L 27/56; A61K 35/50; C12N 5/071; C12N 5/0775; C12N 5/077

(52) U.S. Cl. CPC A61L 27/3604; A61L 27/3687; A61L 27/56; A61L 2430/34; C12N 5/0697; C12N 5/0662; C12N 5/0656; A61K 35/50

(57) ABSTRACT

The present invention provides a lyophilized placental composite sheet as a tissue graft for wound care and a method for preparing the lyophilized placental composite sheet. The lyophilized placental composite sheet includes an amniotic membrane and a processed chorion layer for treating various types of wounds and tissue regenerative processes.

This application claims the benefit of U.S. provisional application No. 62/451,361 filed Jan. 27, 2017, the entire content of which is expressly incorporated herein by reference thereto.

### FIELD OF THE INVENTION

The present invention discloses a lyophilized placental composite sheet as a tissue graft for wound care and a method for preparing the lyophilized placental composite sheet.

### BACKGROUND OF THE INVENTION

Human skin wounds cost the American healthcare system billions of dollars by affecting millions of patients every year (Sen et al., Human skin wounds: a major and snowballing threat to public health and the economy, Wound Rep and Reg, November 2009, 17(6), page 763-771). The skin wound could result from surgery, trauma, diabetes, pressure, vascular insufficiency, burns, necrotizing soft tissue, or vasculitis (Degreef et al., How to heal a wound fast, Dermatol Clin 1998; 16: 365-375). Non-healing chronic wounds are a growing problem with an incidence of 5 to 7 million cases per year in the United States (Hanson et al., Mesenchymal stem cell therapy for nonhealing cutaneous wounds. Plast Reconstr Surg, 2010, 125, page 510-516).

Wound healing is a complex process with a series of coordinated events which occur in three overlapping phases, i.e., inflammatory, proliferative and remodeling, with the involvements of cells, growth factors, and extracellular matrix proteins coordinated by the endogenous mesenchymal stem cells. Remodeling is the final phase of wound healing, which may last 1 to 2 years or even longer involving degradation and synthesis of fibronectin and collagen with increased collagen deposition and new matrix accumulation. (Maxson et al., Concise review: role of mesenchymal stem cells in wound repair, Stem Cell Translational Medicine, 2012, 1:142-149)

There are growing studies demonstrating the advantages of using exogenous mesenchymal stem cells to coordinate repair responses by recruiting cells, growth factors and extracellular matrix proteins in wound repair. Preferred wound-healing products should have compositions resembling the components in skin including particular growth factors, extracellular matrix proteins, viable epithelial cells, fibroblasts and mesenchymal stem cells (Maxson et al.).

Historically, human placenta tissues have been used in medicine since 1910, and studies have been done to investigate the use of cryopreserved human amniotic membrane as a surgical patch in immunologic unprivileged anatomic sites (abstract, Kesting et al., Cryopreserved human amniotic membrane for soft tissue repair in rats, Ann Plast Surg, June 2008, 60(6), page 684-691). Both amniotic and chorionic membranes have been used as skin substitutes for wound treatments. Various proteins which are beneficial for wound healing include physiological growth factors, anti-inflammatory factors, antimicrobial factors, angiogenic proteins, epithelial cell stimulatory proteins, and anti-scarring proteins (page 147 and table 2, Maxson et al.).

Amnion (amniotic membrane) is the innermost membrane that closely covers the embryo when first formed. In general, when an amnion is isolated from fresh placenta, it is freed from the connective tissue of the umbilical cord and trophoblast tissues and contains epithelium, basement membrane, compact layer, fibroblast layer and spongy layer. Amnion contains various biologic factors, such as cytokines, epidermal growth factor, transforming growth factor, collagen, laminin and fibronectin. Amnion has been used to promote cell growth for wound healing and exhibits the effects of anti-inflammation, anti-angiogenesis, anti-fibrotic response and anti-microbial activities.

Chorion (chorionic membrane) is one of the membranes that exist during pregnancy between the developing fetus and mother, and is the outermost membrane surrounding an embryo, which contributes to the formation of the placenta.

Baur (U.S. Pat. No. 4,361,552, Wound dressing) discloses a wound dressing comprising an amnion in which the proteins have been fixed by cross-linking. Kinoshita et al. (U.S. Pat. No. 8,231,908 B2, Sheet-like composition) discloses a sheet-shaped composition comprising an amnion having its epithelial layer removed, in which the modified amnion is lyophilized and trehalose-treated. Daniel et al. (U.S. Pat. No. 9,186,382 B2, Placental tissue grafts produced by chemical dehydration/freeze-drying and methods for making and using the same) discloses placental tissue grafts comprising amnion and/or chorion, which are produced by chemical dehydration following by freeze-drying. Samaniego (U.S. Pat. No. 9,480,549 B2, Multi-layer tissue patches) discloses wound dressings comprising a multi-layer amnion tissue patch which is treated with glutaraldehyde. Koob et al. (US 2014/0271728 A1, Molded placental tissue compositions and methods of making and using the same) discloses molded dehydrated placental tissue compositions with defined size and shape comprising micronized amnion, chorion or placental tissues. McQueen et al. (US 2016/0067287 A1, Micronized placental tissue compositions with optional sealant and methods of making and using the same) discloses micronized placental components including a sealant, such as adhesive or gelation agent.

Various wound-healing products for clinical uses including bioengineered dressings and cell-based products have been made. The performances of these products are not optimal, however, and the wound healing remains an unmet medical need. The present invention now addresses these needs and provides viable improvements that have not been previously disclosed in the art.

### SUMMARY OF THE INVENTION

The present invention now provides methods for preparing pre-grafts and tissue grafts for wound care along with the resulting pre-grafts and tissue grafts obtainable from the methods.

The method for preparing a tissue graft for wound care comprises providing an amniotic membrane that has an epithelial layer on one surface and a spongy layer on the opposite surface; applying chorion pieces or particles onto the spongy layer of the amniotic membrane to form a pre-graft; contacting the chorion pieces or particles with a treatment solution that includes a first lyoprotectant; and freeze-drying the pre-graft to form the tissue graft as a composite sheet. The treatment solution advantageously comprises water and the lyoprotectant in an amount sufficient to maintain or preserve biologic activities and structure of the chorion pieces or particles during freeze-drying to facilitate formation of the tissue graft. Also, the chorion pieces or particles are applied in an amount sufficient to form a processed chorion layer on the spongy layer of the amniotic membrane after freeze-drying wherein the chorion layer mimics or preserves native chorion properties or structure.

The method includes spreading the amniotic membrane over a support and applying the chorion pieces or particles onto the amniotic membrane while it is on the support. The chorion pieces and/or particles are preferably applied from a mixture such as a slurry that is also homogenized and are applied in a substantially even distribution on the amniotic membrane. The mixture comprises the chorion pieces or particles and the treatment solution. Also, the pre-graft is preferably stored at a freezing temperature prior to freeze-drying the pre-graft. Typically, the composite sheet is cut to one or more desired sizes and aseptically packaged.

The first lyoprotectant is typically selected from the group consisting of diffusible cryoprotectors, non-diffusible cryoprotectors, polyol cryoprotectors, and combinations thereof, while the treatment solution generally also includes a lyoprotectant bulking agent in an amount sufficient to maintain or preserve tissue structure in the tissue graft, and a lyoprotectant binding agent in an amount sufficient to help attach the chorion pieces or particles to the spongy layer during freeze-drying. The lyoprotectant is diffusible cryoprotectors, including dimethyl sulfoxide (DMSO), glycerol, 1,2-propanediol, 2,3-butanediol, and polyethylene glycol; non-diffusible cryoprotectors, including polyvinylpyroldone, hydroxyl starch, and sugars; polyol cryoprotectors, including trehalose, raffinose, sucrose, mannitol, lactose, glucose, maltose, maltotriose, maltotetraose, maltopentaose, maltoheptaose, dextran 1060 (dextran with average molecular weight 1060), detran 4900 (dextran with average molecular weight 4900), and dextran 10200 (dextran with average molecular weight 10200); stabilizers, including sucrose, trehalose, glucose, lactose, maltose, and other disaccharides; tonicity adjusters, including mannitol, sucrose, glycine, glycerol, and sodium chloride; bulking agents, including mannitol, sucrose, and other disaccharides; or combinations thereof.

The invention also provides a tissue graft for wound care obtainable by one of the methods disclosed herein. Typically, this tissue graft comprises a freeze-dried amniotic membrane that has an epithelial layer on one surface and a spongy layer on the opposite surface, wherein the spongy layer includes a layer of freeze-dried chorion pieces or particles.

The invention also provides a pre-graft for preparing a tissue graft for wound care comprising an amniotic membrane that has an epithelial layer on one surface and a spongy layer on the opposite surface, and chorion pieces or particles on the spongy layer, wherein the chorion pieces or particles are treated by a treatment solution comprising water and a first lyoprotectant in an amount sufficient to preserve biologic activities and structure of the chorion pieces or particles during freeze-drying to facilitate formation of the tissue graft; and wherein the chorion pieces or particles are present in an amount sufficient to form a processed chorion layer on the spongy layer of the amnion membrane after freeze-drying. The treatment solution may include multiple lyoprotectants including a lyoprotective bulking agent in an amount sufficient to maintain or preserve tissue structure in the tissue graft during freeze-drying, and a lyoprotectant binding agent in an amount sufficient to help attach the chorion pieces or particles to the spongy layer during freeze-drying.

Also, the invention provides a tissue graft prepared by freeze-drying the pre-graft.

The tissue graft of the present invention can be used for wound care wherein the wound results from surgery, trauma, diabetes, pressure, vascular insufficiency, burns, necrotizing soft tissue, or vasculitis.

### BRIEF DESCRIPTION OF THE DRAWING FIGURES

Further features of the inventive concept, its nature and various advantages will be more apparent from the following detailed description, taken in conjunction with the accompanying figures:

FIG. 1 shows the various lyophilized placental composite sheets prepared using different treatment solutions.

FIG. 2 shows the measurements of hepatocyte growth factor (HGF) levels in lyophilized placental composite sheets prepared using different lyoprotectants compared to HGF levels in normal tissue. The lyoprotectant(s) were prepared in phosphate buffered saline (PBS) solutions.

FIG. 3 shows the measurements of hepatocyte growth factor (HGF) levels in lyophilized placental composite sheets prepared with or without the preferred treatment solution that includes the lyoprotectant combination of mannitol, trehalose and glycerol.

FIG. 4 shows the analysis results in comparing lyophilization and heat-drying of placental composite sheets in the preservation of growth factors.

FIG. 5 Human dermal fibroblasts (FIG. 5A, 5B) and mesenchymal stem cells (MSCs) (FIG. 5C, 5D) were seeded for 24 hours on biopsies of lyophilized placental composite sheets and then stained with a green dye, Calcein AM (acetoxymethyl), to highlight viable cell numbers. Cells were imaged at 4× (FIG. 5A, 5C) and 10× (FIG. 5B, 5D) magnification showing the ability of the two cell types to attach and maintain viability. FIG. 5E shows that fibroblasts were used to compare cell proliferation by seeding fibroblast cells in wells containing lyophilized placenta treated media versus control wells containing non-treated media with no added growth factors.

FIG. 6 shows the comparison of the bioactivity of preserved tissue components. Mesenchymal stem cells and fibroblasts were seeded on biopsies of either donor matched lyophilized or oven-dried composite tissue in a 96-well plate.

FIG. 7 shows the comparison between the lyophilized placental composite sheet and the placental patches of a leading commercial product for wound healing. The levels of growth factors and extracellular matrix (ECM) components were quantified and compared.

FIG. 8 shows that human dermal fibroblasts were seeded in vitro to compare the effectiveness of the lyophilized placental composite sheets to stimulate cell migration against the placental patches of the a leading commercial product for wound healing.

### DETAILED DESCRIPTION OF THE INVENTION

Throughout this description, the preferred embodiments and examples provided herein should be considered as exemplar, rather than as limitations of the present invention.

The present invention provides a lyophilized placental composite sheet as a tissue graft for wound care, which comprises an amniotic membrane and processed placental tissues (e.g. particulated placental tissues, placental tissue mixture, or placental tissue slurry) for treating various types of wounds and tissue regenerative processes, and may exhibit the effects of anti-inflammation, anti-angiogenesis, anti-fibrotic response and anti-microbial activities. In one preferred embodiment, the processed placental tissue is chorion.

In one embodiment, a lyophilized placental composite sheet that comprises an intact amniotic membrane and processed chorionic membrane is prepared. The amniotic and chorionic membranes are isolated from fresh placenta and are treated with a treatment solution containing a lyoprotectant. The placenta used in the present invention can be obtained from any mammal, such as pigs, cows, dogs, sheep, or goats, or preferably from humans. The chorionic membrane is chopped into smaller pieces, typically of the sizes of 1 mm or less. Alternatively, the chorionic membrane can be and preferably is particulated using a tissue homogenizer to obtain a slurry of chorionic membrane particles. The particles and pieces can also be mixed together.

Optionally, the chorionic membrane mixture or slurry may also contain amniotic membrane slurry, placental tissue slurry, trophoblast tissues, or umbilical cord tissue slurry. The chorionic membrane mixture includes predominantly the chorion pieces or particles (i.e., greater than 50% by weight), and typically of at least 60 to 80% by weight. If sufficient chorionic membrane is available, the mixture or slurry can include only chorion pieces or particles.

One reason for adding different tissue with the chorion pieces or particles is because there often is insufficient amounts of chorion available from the harvested placenta. As the final tissue grafts are cut to predetermined sizes, any left-over or unusable sizes of tissue material can be collected and particularized with the chorion membrane to increase the amount of tissue material in the mixture. Of course, if sufficient amounts of chorion membrane is available, the entire mixture can include only chorion tissue. And the additional tissue to be added preferably excludes blood vessels or any potential immunogenic components. The resulting chorion mixture or slurry can be deposited to form a chorion layer after freeze drying that mimics the native structure and properties of the initial chorionic membrane.

To assemble a placental composite sheet, the amniotic membrane is spread over a non-stick surface, and the chorionic membrane mixture or slurry is spread evenly over the amniotic membrane. The placental composite sheet is then subject to a freeze-drying (lyophilizing) process.

The lyophilized placental composite sheet of the present invention produces a pliable but durable felt-like graft after lyophilization and has superior handling properties, which is differentiable and superior in the texture and quality compared to other products in the marketplace. The flexibility and durability of the lyophilized placental composite sheet of the present invention allows the user to easily apply the placental composite sheet as a graft to a wound site, while the softer physical characteristics allow for easy integration of the placental tissues into the wound. In one preferred embodiment, the surface of the lyophilized placental composite sheet of the present invention containing the processed placental tissues is used to contact the wound site. The rehydrated processed placental tissues form a paste-like texture which allows the superior integration of the placental tissues into the wound.

The amnion of the placental composite sheet of the present invention may contain various layers, such as epithelium, basement membrane, compact layer, fibroblast layer, spongy layer, or the combinations thereof. Generally, the chorion pieces or particles are obtained by removing the chorionic membrane from the placental tissue that is obtained. In one embodiment, the presence of the epithelium layer of the amnion in the lyophilized placental composite sheet of the present invention provides advantages of improving wound healing. The surface of spongy layer is sticky and rough, and the presence of exposed spongy layer of the amnion facilitates the receipt of the processed chorion layer thereon for assembly of the placental composite sheet. When the mixture or slurry of chorion pieces or particles is applied onto the surface of spongy layer, the resultant composite sheet has better adhesion compared to other layers of the amnion.

The presence of a first lyoprotectant in the treatment solution used in the process of preparing the placental composite sheet provides the advantages of preserving the biologic activities of native proteins, compounds and matrix in placental tissues compared to other lyophilization processes. In one preferred embodiment, the treatment solution contains one or more lyoprotectants, most preferably selected from mannitol, trehalose, glycerol, or combinations thereof, to treat the processed placental tissues of the placental composite sheet.

Regarding the physical characteristics, the presence of multiple lyoprotectants are also advantageous in assembling the placental composite sheet by facilitating the formation of placental tissue mixture or slurry (particulated placental tissues) having a sufficient consistency or viscosity to be able to spread and stick onto the amnion sheet. The presence of a lyoprotectant solution in a sufficient amount facilitates having the placental tissue particles being together by keeping the placental tissue mixture wet and sticky with increased thickness, not resulting in a powdery or in a flowable form. Due to the use of a lyoprotectant, the placental tissue mixture possesses desired texture and can be more evenly spread over the amnion sheet to obtain even distribution and can be held together during the freeze-drying process. The even distribution of the placental tissue mixture or slurry can be observed by examining the even opaqueness of the placental composite sheet after applying the placental tissue mixture or slurry.

In particular, the combination of the use of lyoprotectant and the presence of exposed spongy layer results a placental composite sheet which has unique desirable handling characteristics during the formation of the placental composite sheet. Therefore, the placental composite sheet can be assembled properly without the addition of sealants, such as adhesives or gelation agents, without the use of any physical or mechanical means, such as molding, or compressing, without the addition of another layer of membrane/substrate on top of the placental tissue slurry, and without the use of fixing/cross-linking agents.

### Configurations of Lyophilized Placental Composite Sheets

The lyophilized placental composite sheet of the present invention comprises amnion and processed placental tissues, wherein the processed placental tissues are placed on the top of a sheet of an amniotic membrane. The amniotic membrane of the placental composite sheet of the present invention may contain various layers, such as epithelium, basement membrane, compact layer, fibroblast layer, spongy layer, or combinations thereof. The processed placental tissues may comprise chorion, amnion, other placental tissues, umbilical cord, or combinations thereof. In one preferred embodiment, the amniotic membrane sheet has a spongy layer on one surface and an epithelium layer on the opposite surface, wherein the processed placental tissues contact the exposed spongy layer. In one preferred embodiment, the processed placental tissue is chorion. The chorion and placental tissues are treated with a lyoprotectant solution prior to assembling the placental composite sheet. In one preferred embodiment, the lyoprotectant solution comprises a mixture of mannitol, trehalose and glycerol as described further herein. Also, application of the mixture or slurry onto the amniotic membrane also introduces treatment solution into the membrane to facilitate freeze drying of the overall composite sheet.

### Lyophilization of Placental Composite Sheets

Removal of water from biologically active products, such as tissue grafts, is important to preserve the functionalities of the products by preventing long term degradation of important biologic molecules. Drying with high temperatures (such as oven-drying) successfully desiccates materials, but it can damage or destroy heat sensitive components of a tissue product. In addition, when a tissue is dried from the liquid state, it will shrink and become relatively insoluble leading to permanent chemical alteration. Lyophilization or freeze-drying solves this problem by freezing tissue to immobilize the water to prevent shrinkage and by using low temperatures and vacuum pressure to remove water. However, freeze-drying has its own challenges, such as protein instability. In order to avoid damaging tissue structures and establishing suitable physical properties, lyoprotectant solutions are often used to maintain structures and prevent denaturation of proteins as water is removed.

The research conducted in the effects of freeze-drying on virus and biologic compounds uses objective measurements to define various parameters of the freeze-drying process. However, the mechanisms of the preservation of tissues using freeze-drying are unclear. Certain freeze-drying processing steps can have deleterious effects on both structure and function of the tissues (William Tomford, Musculoskeletal Tissue Banking, 1993, Raven Press. Ltd., pages 193-194, The effects of freeze-drying on tissues).

The factors effecting the physical characteristics of the tissue during freeze-drying may or may not have impact on its biologic properties. A freeze-drying process designed to maintain the biomechanical strength of an intact tissue may be different from the freeze-drying process of preserving proteins of tissue powder (page 194, Tomford, Musculoskeletal Tissue Banking). Different tissues and cells react differently to the freeze-drying process. The optimal conditions for freeze-drying tissue particles and intact tissues could be very different.

The present invention provides a unique method to prepare a tissue composite sheet comprising both tissue particles and intact tissue membrane through freeze-drying. It is desirable that the resulted lyophilized placental composite sheet has superior physical characteristics, such as flexibility, durability, and softness, and still retains the biologic activities of the components in the placental tissues (such as, proteins, liposomes, matrix and compounds). In particular, the placental composite sheet has the desired integrity of adhering the placental tissue particles to the amniotic membrane during assembling and freeze-drying, and the resulted lyophilized placental composite sheet have excellent physical characteristics by avoiding a composite sheet which is thick, brittle and powdery.

### Lyoprotectant

Various cryoprotective substances (cryoprotectors) can be used as lyoprotectants to protect proteins, cells, liposomes or tissues during freeze-drying without fully understanding their mechanisms of action. The inhomogeneous diffusion of the cryoprotector throughout the thickness of the tissue is a concern for designing a freeze-drying process. Some cryoprotectors can be classified into two categories: including diffusible cryoprotectors with relatively lower molecular weights, which can cross the cell membrane, such as dimethyl sulfoxide (DMSO), glycerol, and 1,2-propanediol; and non-diffusible cryoprotectors with relatively higher molecular weights, which do not cross cell membrane, such as polyvinylpyroldone, hydroxyl starch, and certain sugars (such as, trehalose or sucrose) (page 121, Joseph Bakhach, The cryopreservation of composite tissues, Organogenesis, volume 5, issue 3, 2009, pages 119-126). Some polyols (such as saccharides, sugar alcohols, or dextrans) with various molecular weights demonstrate protective effects against freeze-drying-induced structural perturbation of proteins. The polyols may act by replacing essential water molecules through molecular interaction with proteins to protect protein conformation against dehydration stresses, therefore retains the physical and chemical stability of proteins during freeze-drying process (Izutsu et al., Protection of protein secondary structure by saccharides of different molecular weights during freeze-drying, Chem. Pharm. Bull. 52(2), pages 199-203, 2004). Stabilizers, such as sucrose, trehalose, glucose, lactose, maltose or other disaccharides, may act as stabilizers by forming an amorphous sugar glass to stabilize liposomes and proteins during freeze-drying or reducing proteins by means of the maillard reaction due to the reducing power of certain sugars. Tonicity adjusters, such as mannitol, sucrose, glycine, glycerol, and sodium chloride may be added to the freeze-drying process to maintain an isotonic formulation. A lyoprotective bulking agents, such as mannitol, sucrose, or other disaccharides, may be added to the freeze-drying process to maintain or preserve mechanical properties by providing bulk to the tissues. (Bedu-Addo, Understanding lyophilization formulation development, Pharmaceutical Technology, Lyophilization, 2004, pages 10-18)

The examples of lyoprotectants comprise: diffusible cryoprotectors, including dimethyl sulfoxide (DMSO), glycerol, 1,2-propanediol, 2,3-butanediol, and polyethylene glycol; non-diffusible cryoprotectors, including polyvinylpyroldone, hydroxyl starch, and sugars; polyol cryoprotectors, including trehalose, raffinose, sucrose, mannitol, lactose, glucose, maltose, maltotriose, maltotetraose, maltopentaose, maltoheptaose, dextran 1060 (dextran with average molecular weight 1060), detran 4900 (dextran with average molecular weight 4900), and dextran 10200 (dextran with average molecular weight 10200); stabilizers, including sucrose, trehalose, glucose, lactose, maltose, and other disaccharides; tonicity adjusters, including mannitol, sucrose, glycine, glycerol, and sodium chloride; bulking agents, including mannitol, sucrose, and other disaccharides.

A lyoprotectant solution is used to prepare the lyophilized placental composite sheet of the present invention, wherein the lyoprotectant is preferably selected from the group consisting of diffusible cryoprotectors, non-diffusible cryoprotectors, and polyol cryoprotectors.

### Treatment Solution

The treatment solution of the present invention includes one or more and preferably a combination of the lyoprotectants disclosed herein. Advantageously, a lyoprotectant bulking agent is present in an amount sufficient to maintain or preserve tissue structure in the tissue graft, and a lyoprotectant binding agent is present in an amount sufficient to help attach the chorion pieces and/or particles to the spongy layer during freeze drying.

The lyoprotectant may be selected from diffusible cryoprotectors, including dimethyl sulfoxide (DMSO), glycerol, 1,2-propanediol, 2,3-butanediol, and polyethylene glycol; non-diffusible cryoprotectors, including polyvinylpyroldone, hydroxyl starch, and sugars; polyol cryoprotectors, including trehalose, raffinose, sucrose, mannitol, lactose, glucose, maltose, maltotriose, maltotetraose, maltopentaose, maltoheptaose, dextran 1060 (dextran with average molecular weight 1060), detran 4900 (dextran with average molecular weight 4900), and dextran 10200 (dextran with average molecular weight 10200); stabilizers, including sucrose, trehalose, glucose, lactose, maltose, and other disaccharides; tonicity adjusters, including mannitol, sucrose, glycine, glycerol, and sodium chloride; bulking agents, including mannitol, sucrose, and other disaccharides. Various combinations can be used with preferred combinations disclosed herein.

Generally, a combination of multiple lyoprotectants is present in the treatment solution. The first lyoprotectant is present in an amount of up to 12% (w/v), a lyoprotectant bulking agent is present in in an amount of up to 30% (w/v), and a lyoprotective binding agent present in an amount of up to 6% (w/v). In these solutions, water represents the balance and is present in an amount of between 52 and 98% (w/v).

Preferably, the first lyoprotectant is a disaccharide and is present in an amount of between 0.2 and 8% (w/v), the lyoprotectant bulking agent is a sugar and is present in an amount of between 0.5 and 20% (w/v), the lyoprotectant binding agent is a C2-C6 alcohol or polyol having two to four hydroxyl groups and is present in an amount of between 0.1 and 4% (w/v), and the water is present in an amount of between 75 and 95% (w/v). In a more preferred treatment solution, the first lyoprotectant is trehalose and is present in an amount between 0.4 and 4% (w/v), typically 2%, the lyoprotectant bulking agent is mannitol and is present in an amount between 1 and 12% (w/v), typically, 6%, the lyoprotectant binding agent is glycerol and is present in an amount of between 0.2 and 2% (w/v), typically 1%, and the water is present in an amount of between 75 and 95% (w/v).

The treatment solution composed of these ingredients preserves the native proteins and matrix of the final tissue grafts compared to other dehydration processes. It also results in unique handling characteristics of the tissue grafts that are markedly different from existing products. Accordingly, these tissue graft sheets are designed for use in treating various types of wounds or tissue regenerative processes.

### EXAMPLES

The features and improved properties of these tissue grafts are shown in the examples which illustrate the benefits and advantages of the present invention.

### Example 1. Preparation of a Lyophilized Placental Composite Sheet

A lyophilized placental composite sheet that comprised an intact amniotic membrane and processed chorionic membrane was prepared.

Fresh placentas were harvested and aseptically packaged with ice blocks and shipped to manufacturing site. Within 72 hours of birth, aseptic process technicians manually isolated the amniotic and chorionic membranes from the fresh placenta. These membranes were manually cleaned with several washes in a sterile saline and citrate dextrose solution (anti-coagulant). Blood and trophoblast tissues were removed during the cleaning process. The isolated amniotic membrane was freed from the connective tissue of the umbilical cord and trophoblast tissues. The cleaned membranes were then further treated with a mild disinfectant or antibiotic cocktail solution. After rinsing off the disinfectant, these membranes were further processed by immersing them in a lyoprotectant solution. The chorionic membrane was further particulated using a tissue homogenizer to obtain a chorionic membrane mixture or slurry. Optionally, the chorionic membrane mixture or slurry may contain amniotic membrane slurry or placental tissue slurry. To assemble a placental composite sheet, the amniotic membrane was spread over a non-stick surface. Then, the chorionic membrane mixture or slurry was spread evenly over the amniotic membrane. Dry ice blocks were used to freeze the resulted placental composite sheet. The frozen placental composite sheet may optionally be stored at a freezing temperature that is preferably less than or equal to −60° C. The frozen placental composite sheet then underwent a conventional freeze-drying process. The lyophilized placental composite sheet was then cut to size and aseptically packaged into final product.

### Example 2. Physical Characteristics of Lyophilized Placental Composite Sheets

The lyophilized placental composite sheets were prepared according to the method described in Example 1 and were analyzed for their physical and handling properties.

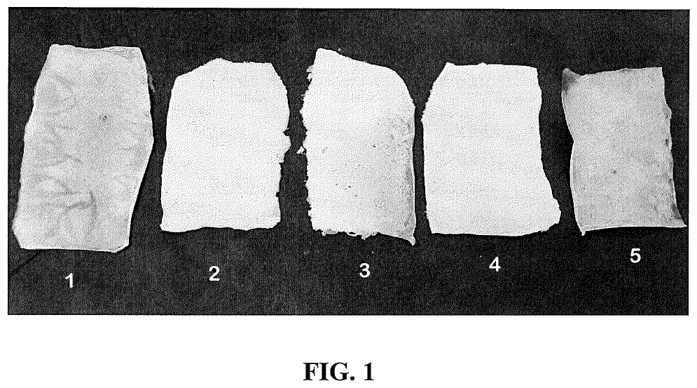

Various lyophilized placental composite sheets were prepared in the presence of water alone (sample 1 of FIG. 1) or with different treatment solutions. The different solutions include: 6% mannitol and 2% trehalose in water (sample 2 of FIG. 1); 6% mannitol and 2% trehalose in PBS (sample 3 of FIG. 1); 6% mannitol, 2% trehalose and 1% glycerol in water (sample 4 of FIG. 1); and 1% glycerol in water (sample 5 of FIG. 1).

When the lyophilized placental composite sheet was prepared in water without lyoprotectant, it appeared as a flat, thin and brittle membrane (sample 1 of FIG. 1). When the lyophilized placental composite sheet was prepared with only 1% glycerol in water, it appeared as a thin and uneven membrane (sample 5 of FIG. 1). When the lyophilized placental composite sheet was prepared in the presence of a treatment solution containing mannitol and trehalose in water, it possessed an improved, thick, felt-like consistency that had much less residue but was somewhat brittle (sample 2 of FIG. 1). Surprisingly, when the lyophilized placental composite sheet was prepared with the most preferred treatment solution of 6% mannitol, 2% trehalose, and 1% glycerol in water (sample 4 of FIG. 1), it had a uniform lyophilized cake with minimal residues, with superior physical and handling characteristics in the categories of flexibility, durability, and softness, and with a pliable and felt-like texture. These results show that combinations of different ingredients in the treatment solution leads to the best properties in the final product, although using different (typically higher) amounts of the individual components can lead to useful tissue graft products. The preferred treatment solutions provide optimum properties with smaller amounts of the solution components.

### Example 3. Preservation of Growth Factors in the Lyophilized Placental Composite Sheets

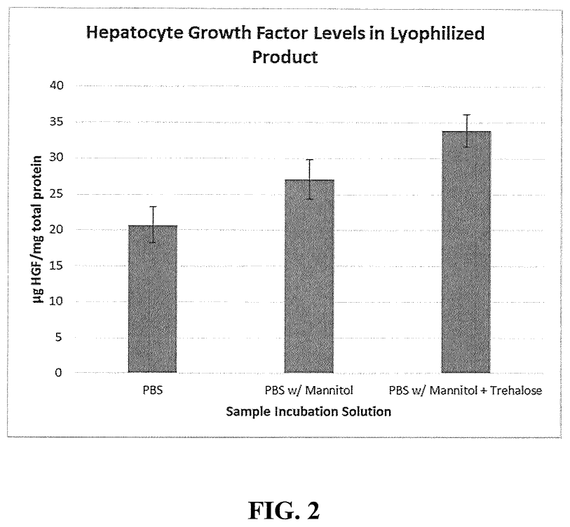

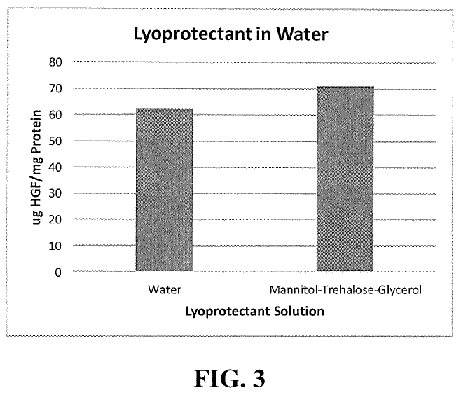

Hepatocyte growth factor (HGF) levels were measured in lyophilized placental composite sheets prepared using various lyoprotectant solutions and compared to the HGF levels in normal tissue. The lyophilized placental composite sheet prepared with the lyoprotectant solution containing both mannitol and trehalose preserved the greatest amounts of HGF compared to PBS alone (FIG. 2). Furthermore, when the lyophilized placental composite sheet was prepared with 6% mannitol, 2% trehalose and 1% glycerol in water, the resulting tissue graft exhibited better preservation of HGF as compared to water alone (FIG. 3).

### Example 4. Comparison of Lyophilization and Heat-Drying of Placental Composite Sheets in the Preservation of Growth Factors

Placental composite sheets were prepared according to method described in Example 1 and their properties were analyzed. Placental membranes were cleaned and processed. A chorionic membrane slurry was spread evenly over a rectangular section of amniotic membrane to assemble a placental composite sheet. Two placental composite sheets were prepared from each placental donor. One placental composite sheet was lyophilized using a conventional freeze-drying process, while the other placental composite sheet was prepared using heat-drying (oven dried) in an incubator oven at 37° C. The lyophilized placental composite sheet was prepared using a lyoprotectant comprising 6% mannitol, 2% trehalose and 1% glycerol in water.

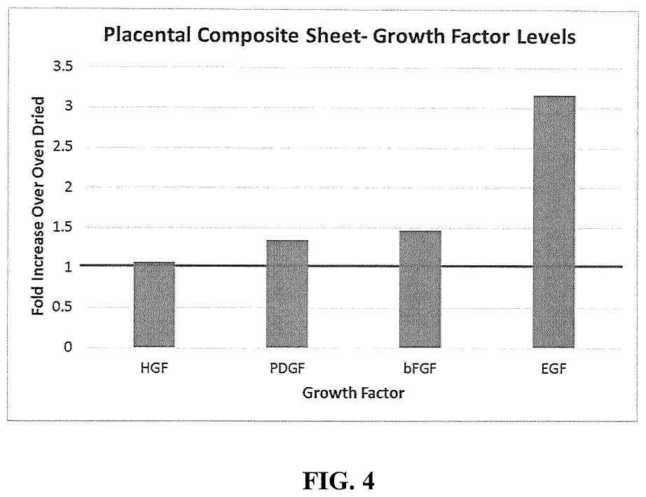

Growth factors are some of the proteins which are most sensitive to degradation during processing and storage. During the removal and processing of fresh placentas, specific combinations of preservation and freeze-drying methods were evaluated and compared to heat-drying methods by quantifying levels of growth factors which were critical or important to wound healing. Following each drying method, the placental composite sheets were analyzed to measure different levels of these growth factors using ELISA (enzyme-linked immunosorbent assay). Quantities of growth factors in lyophilized placental composite sheets were normalized to oven-dried (heat-drying) samples with matching donor. The analysis results were reported as fold changes in protein levels. The results indicated that the lyophilization methods preserved greater levels of hepatocyte growth factor (HGF), platelet-derived growth factor (PDGF), basic fibroblast growth factor (bFGF), and epidermal growth factor (EGF) in the lyophilized placental composite sheets comparing to heat-drying methods with almost a 10-fold combined total increase. As indicated in FIG. 4, EGF has the greatest level in fold increase over oven-dried samples, which is the most sensitive growth factor. These growth factors play important roles in cell migration, proliferation, and maintenance of normal cell phenotype during wound healing.

### Example 5. Lyophilized Placental Composite Sheets Support Cell Attachment, Viability, and Proliferation

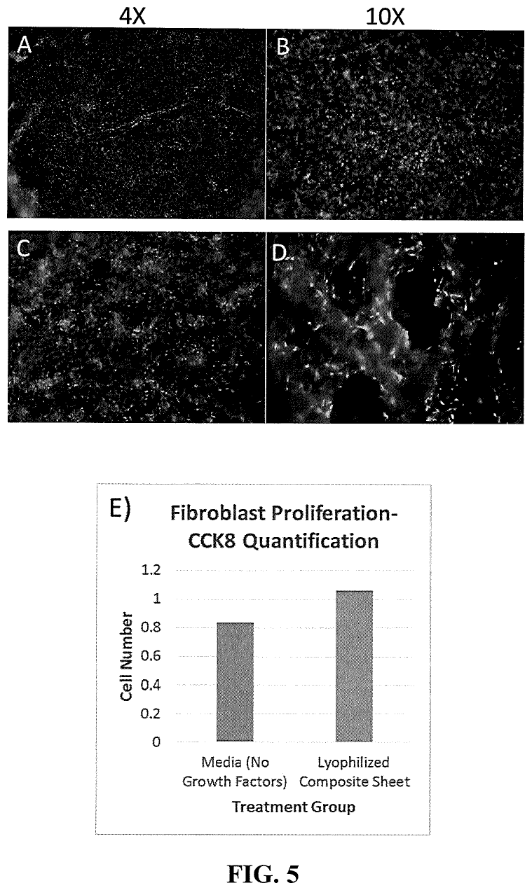

To determine the abilities of the lyophilized placental composite sheet to act as a cell substrate, two cells types, including human mesenchymal stem cells (MSCs) and human dermal fibroblasts which are important to wound healing, were seeded on 10 mm biopsies of the tissue sheets in a 96-well plate. Cells were allowed to attach for 24 hours before calcein AM (acetoxymethyl) was added to the media. Intracellular esterases convert calcein AM to a green fluorescent dye allowing viable cells to be visible. Microscope images were taken of viable fibroblasts (FIG. 5A, 5B) and MSCs (FIG. 5C, 5D) at 4× and 10× magnifications. Background tissue of the placental composite sheet was highlighted in red or blue auto-fluorescence. Staining showed that placental composite sheets effectively supported cell attachment and viability for multiple cell types.

Fibroblasts were also used to compare cell proliferation by seeding fibroblast cells in wells containing lyophilized placenta treated media versus control wells containing non-treated media with no added growth factors. Fibroblast cells seeded at equal concentrations were allowed to grow for 3 days until quantified. To measure cell proliferation, water-soluble tetrazolium salt (WST-8) was added to the media in all wells. The cell number, which is relevant to cell proliferation, was measured through dye quantification of WST-8 reduction related to the dehydrogenase activity of cells. Cells proliferated significantly faster in media treated with lyophilized placental composite sheets versus non-treated media (FIG. 5E).

### Example 6. Lyophilized Placental Composite Sheets Preserve Greater Growth Factor Bioactivity and Support Greater Cell Function Compared to Oven-Dried Tissue

To test and compare the bioactivity of preserved tissue components, MSCs and fibroblasts were seeded on biopsies of either donor matched lyophilized or oven-dried composite tissue in a 96-well plate. Function of these cell types was assessed by quantification of additional growth factors produced by seeded cells. MSCs, which regulate the immune response and inflammation, secreted greater levels of transforming growth factor beta-3 (TGF-β3), an anti-scarring growth factor involved in re-epithelialization of wounds (FIG. 6A). Fibroblasts, which rebuild extracellular matrix (ECM) and are vital to wound contraction, produced higher levels of the proliferative and anti-scarring growth factors, bFGF and HGF, when seeded on lyophilized tissue compared to heat dried biopsies (FIG. 6B).

### Example 7. Lyophilized Placental Composite Sheets Preserve Higher Levels of Growth Factors and ECM Components Versus a Commercial Placental Product

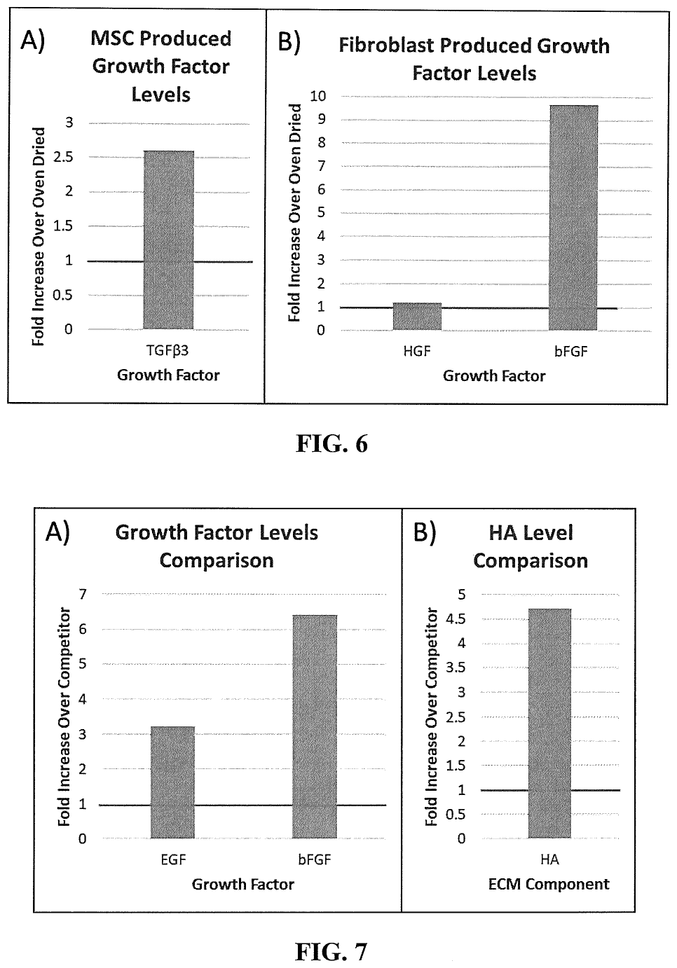

To demonstrate the advantages of the lyophilized placental composite sheet prepared by the processing methods of the present invention in comparison to the placental patches of a well-established commercial product for wound healing, levels of important growth factors and ECM components were quantified and compared. Equal sized 2×2 cm² pieces of the lyophilized placental composite sheets of the present invention and the competitor product were solubilized. The levels of EGF and bFGF were quantified using ELISAs. EGF levels were over 3-fold greater in the lyophilized composite sheets, and bFGF levels were almost 7-fold greater in lyophilized sheets compared to the oven-dried commercial product (FIG. 7A). In a separate assay, the ECM component hyaluronic acid (HA) was quantified using an ELISA and showed an almost 5-fold increase in levels in the lyophilized placental composite sheet compared to the commercial product (FIG. 7B). EGF and bFGF are both potent important mitogens during tissue repair, while bFGF also promotes cell migration and reduces scarring. Hyaluronic acid is unique type of glycosaminoglycan affecting physical properties of the tissue. HA also modulates inflammation and influences cell motility and function in the body.

### Example 8. Lyophilized Placental Composite Sheets According to the Present Invention Promote Greater Cell Migration Compared to a Commercial Placental Product

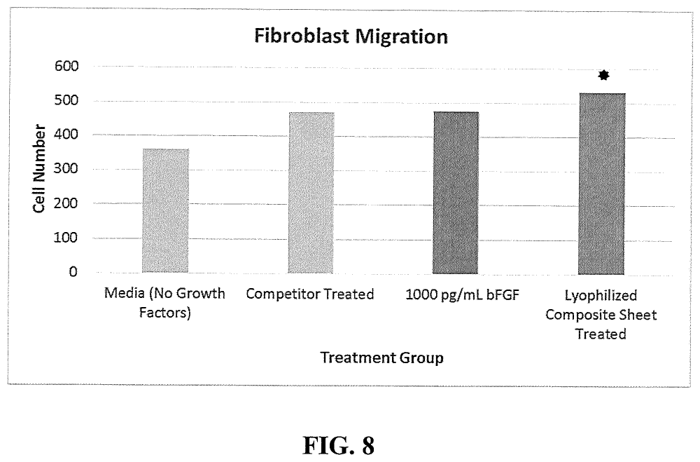

Fibroblasts migrate to wound sites during tissue repair and regrowth. Human dermal fibroblasts were seeded in vitro to compare the effectiveness of the lyophilized placental composite sheets to stimulate cell migration against the placental patches of a currently commercial product for wound healing. A transwell migration assay was performed by seeding cells at a set concentration on top of a permeable transwell mesh. Wells below the mesh insert either contained a biopsy of the lyophilized composite sheets or the commercial product, enabling the biopsies to be incubated in the media. Wells without biopsies containing media with or without growth factors were used as positive and negative controls. Cells in every well were allowed to migrate through the mesh overnight and then stained with DAPI (4′,6-diamidino-2-phenylindole) and imaged. Fibroblasts migrated at significantly higher levels in the presence of the lyophilized composite sheets compared to the controls and commercial product (FIG. 8).

It is to be understood that the present invention is not to be limited to the exact description and embodiments as illustrated and described herein. To those of ordinary skill in the art, one or more variations and modifications will be understood to be contemplated from the present disclosure. Accordingly, all expedient modifications readily attainable by one of ordinary skill in the art from the disclosure set forth herein, or by routine experimentation therefrom, are deemed to be within the true spirit and scope of the invention as defined by the appended claims.

1. A method for preparing a tissue graft for wound care, which comprises:
providing an amniotic membrane that has an epithelial layer on one surface and a spongy layer on the opposite surface;
applying chorion pieces or particles onto the spongy layer of the amniotic membrane to form a pre-graft;
contacting the chorion pieces or particles with a treatment solution that includes a first lyoprotectant; and
freeze-drying the pre-graft to form the tissue graft as a composite sheet;
wherein the treatment solution comprises water and the lyoprotectant in an amount sufficient to maintain or preserve biologic activities and structure of the chorion pieces or particles during freeze-drying to facilitate formation of the tissue graft;
wherein the chorion pieces or particles are applied in an amount sufficient to form a processed chorion layer on the spongy layer of the amniotic membrane after freeze-drying wherein the chorion layer mimics or preserves native chorion properties or structure; and
wherein the amniotic membrane is spread over a support, the chorion pieces or particles are applied onto the amniotic membrane while it is on the support, and the chorion pieces or particles are applied from a homogenized mixture in a substantially even distribution on the amniotic membrane, wherein the mixture comprises the chorion pieces or particles and the treatment solution.

2. The method of claim 1, wherein the treatment solution further comprises additional lyoprotectants including a lyoprotectant bulking agent in an amount sufficient to maintain or preserve tissue structure in the tissue graft, and a lyoprotectant binding agent in an amount sufficient to help attach the chorion pieces or particles to the spongy layer during freeze drying.

3. The method of claim 1, wherein the first lyoprotectant is selected from the group consisting of diffusible cryoprotectors, non-diffusible cryoprotectors, polyol cryoprotectors, and combinations thereof.

4. The method of claim 1, wherein the lyoprotectant is selected from diffusible cryoprotectors, including dimethyl sulfoxide (DMSO), glycerol, 1,2-propanediol, 2,3-butanediol, and polyethylene glycol; non-diffusible cryoprotectors, including polyvinylpyroldone, hydroxyl starch, and sugars; polyol cryoprotectors, including trehalose, raffinose, sucrose, mannitol, lactose, glucose, maltose, maltotriose, maltotetraose, maltopentaose, maltoheptaose, dextran 1060 (dextran with average molecular weight 1060), detran 4900 (dextran with average molecular weight 4900), and dextran 10200 (dextran with average molecular weight 10200); stabilizers, including sucrose, trehalose, glucose, lactose, maltose, and other disaccharides; tonicity adjusters, including mannitol, sucrose, glycine, glycerol, and sodium chloride; bulking agents, including mannitol, sucrose, and other disaccharides; or combinations thereof.

5. A method for preparing a tissue graft for wound care, which comprises:
providing an amniotic membrane that has an epithelial layer on one surface and a spongy layer on the opposite surface;
applying chorion pieces or particles onto the spongy layer of the amniotic membrane to form a pre-graft;
contacting the chorion pieces or particles with a treatment solution that includes a first lyoprotectant, a lyoprotectant bulking agent in an amount sufficient to maintain or preserve tissue structure in the tissue graft, and a lyoprotectant binding agent in an amount sufficient to help attach the chorion pieces or particles to the spongy layer during freeze drying; and
freeze-drying the pre-graft to form the tissue graft as a composite sheet;
wherein the treatment solution comprises water and the lyoprotectant in an amount sufficient to maintain or preserve biologic activities and structure of the chorion pieces or particles during freeze-drying to facilitate formation of the tissue graft, wherein the first lyoprotectant is present in an amount of up to 12% (w/v), the lyoprotectant bulking agent present in an amount of up to 30% (w/v), the lyoprotectant binding agent is present in an amount of up to 6% (w/v), and the water represents the balance and is present in an amount of between 52 and 98% (w/v); and
wherein the chorion pieces or particles are applied in an amount sufficient to form a processed chorion layer on the spongy layer of the amniotic membrane after freeze-drying wherein the chorion layer mimics or preserves native chorion properties or structure.

6. The method of claim 5, wherein the first lyoprotectant is a disaccharide and is present in an amount of between 0.2 and 8% (w/v), the lyoprotectant bulking agent is a sugar and is present in an amount of between 0.5 and 20% (w/v), the lyoprotectant binding agent is a C2-C6 alcohol or polyol having two to four hydroxyl groups and is present in an amount of between 0.1 and 4% (w/v), and the water is present in an amount of between 75 and 95% (w/v).

7. The method of claim 6, wherein the first lyoprotectant is trehalose and is present in an amount between 0.4 and 4% (w/v), the lyoprotectant bulking agent is mannitol and is present in an amount between 1 and 12% (w/v), and the lyoprotectant binding agent is glycerol and is present in an amount of between 0.2 and 2% (w/v), and the water is present in an amount of between 75 and 95% (w/v).

8. The method of claim 6, wherein the first lyoprotectant is trehalose present in an amount of about 2% (w/v); the lyoprotectant bulking agent is mannitol present in an amount of about 6% (w/v); the lyoprotectant binding agent is glycerol present in an amount of about 1% (w/v), and the water is present in an amount of between 75 and 95% (w/v).

9. The method of claim 1 which further comprises storing the pre-graft at a freezing temperature prior to freeze-drying the pre-graft.

10. The method of claim 1 which further comprises cutting the composite sheet to one or more desired sizes and aseptically packaging the cut sheet.

11. A tissue graft for wound care obtainable by the method of claim 1.

12. A tissue graft for wound care comprising a freeze-dried amniotic membrane that has an epithelial layer on one surface and a spongy layer on the opposite surface wherein the spongy layer includes a layer of freeze-dried chorion pieces or particles, a first lyoprotectant in an amount sufficient to maintain or preserve biologic activities and structure of the chorion pieces or particles during freeze-drying to facilitate formation of the tissue graft, a lyoprotectant bulking agent in an amount sufficient to maintain or preserve tissue structure in the tissue graft, and a lyoprotectant binding agent in an amount sufficient to help attach the chorion pieces or particles to the spongy layer during freeze drying.

13. A pre-graft for preparing a tissue graft for wound care comprising an amniotic membrane that has an epithelial layer on one surface and a spongy layer on the opposite surface; and chorion pieces or particles on the spongy layer, wherein the chorion pieces or particles are treated by a treatment solution comprising water and a first lyoprotectant in an amount sufficient to preserve biologic activities and structure of the chorion pieces or particles during freeze-drying to facilitate formation of the tissue graft, a lyoprotectant bulking agent in an amount sufficient to maintain or preserve tissue structure in the tissue graft, and a lyoprotectant binding agent in an amount sufficient to help attach the chorion pieces or particles to the spongy layer during freeze drying; and wherein the chorion pieces or particles are present in an amount sufficient to form a processed chorion layer on the spongy layer of the amnion membrane after freeze-drying.

14. A tissue graft prepared by freeze-drying the pre-graft of claim 13.

15. The method of claim 5 which further comprises storing the pre-graft at a freezing temperature prior to freeze-drying the pre-graft.

16. The method of claim 5 which further comprises cutting the composite sheet to one or more desired sizes and aseptically packaging the cut sheet.

17. A tissue graft for wound care obtainable by the method of claim 5.

18. The tissue graft of claim 11, wherein the lyoprotectant is selected from diffusible cryoprotectors, including dimethyl sulfoxide (DMSO), glycerol, 1,2-propanediol, 2,3-butanediol, and polyethylene glycol; non-diffusible cryoprotectors, including polyvinylpyroldone, hydroxyl starch, and sugars; polyol cryoprotectors, including trehalose, raffinose, sucrose, mannitol, lactose, glucose, maltose; maltotriose, maltotetraose, maltopentaose, maltoheptaose, dextran 1060 (dextran with average molecular weight 1060), detran 4900 (dextran with average molecular weight 4900), and dextran 10200 (dextran with average molecular weight 10200); stabilizers, including sucrose, trehalose, glucose, lactose, maltose, and other disaccharides; tonicity adjusters, including mannitol, sucrose, glycine, glycerol, and sodium chloride; bulking agents, including mannitol, sucrose, and other disaccharides; or combinations thereof.

19. The tissue graft of claim 12, wherein the first lyoprotectant is trehalose; the lyoprotectant bulking agent is mannitol; and the lyoprotectant binding agent is glycerol.

20. The pre-graft of claim 13, wherein the first lyoprotectant is a disaccharide and is present in an amount of between 0.2 and 8% (w/v), the lyoprotectant bulking agent is a sugar and is present in an amount of between 0.5 and 20% (w/v), the lyoprotectant binding agent is a C2-C6 alcohol or polyol having two to four hydroxyl groups and is present in an amount of between 0.1 and 4% (w/v), and the water is present in an amount of between 75 and 95% (w/v).
